(19) United States
(12) Patent Application Publication
(10) Pub. No.: US 2023/0404522 A1
(43) Pub. Date: Dec. 21, 2023

(54) DEVICE AND METHOD FORBREAST CANCER DIAGNOSIS

(71) Applicant: Imedicals S.r.l., Lecce (IT)

(72) Inventor: Sergio Casciaro, Lecce (IT)

(73) Assignee: Imedicals S.r.l., Lecce (IT)

(21) Appl. No.: 18/037,871

(22) PCT Filed: Nov. 24, 2021
(86) PCT No.: PCT/IB2021/060914

§ 371 Date: May 19, 2023

(30) Foreign Application Priority Data
Dec. 1, 2020 (IT) 102020000029309

(51) Int. Cl. A61B 8/08; A61B 8/00; G06T 7/00

(52) U.S. Cl. CPC A61B 8/085; A61B 8/0825; A61B 8/4209; A61B 8/4281; A61B 8/403; A61B 8/406; A61B 8/4455; A61B 8/4477; A61B 8/4494; A61B 8/5253; A61B 8/483; G06T 7/0012; A61B 2576/02; A61B 2562/043; A61B 2562/14

(57) ABSTRACT

An ultrasound device (1) for carrying out a breast diagnostic examination for breast cancer diagnosis, comprising two ultrasound probes, characterized in that each of said probes comprises: a first array of piezoelectric transducers with a first nominal frequency (f1), and a second array of piezoelectric transducers with a second nominal frequency (f2), the probes being arranged opposite to each other, being configured to rotate along a circular trajectory having for center the midpoint of the segment connecting them and to slide to each other along said straight line connecting them, coming in contact with the breast of the patient from diametrically opposite portions, in that the arrays comprise each a plurality of piezoelectric transducers, positioned so that acquisitions are carried out on a plane of acquisition orthogonal to the plane of the circular trajectory.

### BACKGROUND OF THE INVENTION

### 1. Field of the Invention

The present invention relates to a device for carrying out an examination for breast cancer diagnosis.

More in particular, the present invention relates to a device allowing to reconstruct both a 3D ultrasound model of the breast volume and information about the frequency spectra and the acoustic attenuation coefficient in a perfectly overlapping way, and to calculate a diagnostic parameter indicating the presence of a breast carcinoma according to these data.

### 2. Brief Description of the Prior Art

Many problems are linked to the breast ultrasound examination.

In primis, it is desirable to guarantee the repeatability of the examination, and in particular of the ultrasound probe (or probes) positioning with respect to the breast, regardless of the operator manual skills and expertise; in secundis, if the breast is to be 3D reconstrued it is needed to guarantee that the breast is in a well determined position with respect to the ultrasound probe (or ultrasound probes) while this one is moved to scan a plurality of positions, obviously guaranteeing the presence of a suitable acoustic coupling means (water, gel or other suitable material) between the probe and the skin.

Finally, once the above cited technical problems are solved, it is needed to analyze the ultrasound data collected by means of a method allowing high accuracy in breast cancer diagnosis.

Many devices and methods are known for ultrasonic and/or ultrasound data analysis aiming at diagnosing breast cancer, that try to solve said problems.

CN110680380 describes a device using a ring with a plurality of ultrasound transducers arranged thereon, which is positioned outside a cylindrical container inside which the breast of a patient, laying prone, is immersed in water. The device is used to produce ultrasound images according to which the doctor can make a diagnosis of breast cancer.

WO02089672 described an apparatus in which a linear array of piezoelectric transducers is arranged parallel to the axis of symmetry of the breast, and is configured to rotate around the same, inside a container in which a coupling liquid is provided. EP2868279 describes a device for breast ultrasound using a plurality of transducers arranged on a supporting element.

US2013/0041261 describes a device using a ring-shaped transducer, which comprise a plurality of 2 MHz frequency ultrasound senders and receivers, configured to determine a spatial distribution of breast acoustic and mechanical parameters.

US2012029358 shows a device using a plurality of ultrasound probes, arranged symmetrically around the axis of symmetry of the breast, configured for imaging, at frequencies between 7.5 and 10 MHz, in a plurality of planes going through the axis of symmetry of the breast.

US2004064046 described a device for breast ultrasound tomography comprising a stationary chamber for containing a fluid, inside which a mobile chamber is provided, which is integral to an array of ultrasound transducers and receivers.

Other examples are shown in CN111213065, US2013041260, U.S. Pat. No. 4,222,274, U.S. Ser. No. 10/285,667.

### Technical Problem

At the best of the present inventors' knowledge, all the just described devices, and the other ones known at the state of the art, have limits, since they do not allow to realize, during the same examination, a perfectly overlapped volumetric reconstruction both of the breast image and of the distribution of other characteristic parameters of the analyzed tissues, such for example the acoustic attenuation coefficient.

Moreover, no one of the known devices allows to calculate a diagnostic parameter that considers explicitly both piece of information (information obtainable from the ultrasound imaging and information obtainable from the analysis of acoustic attenuation coefficients).

Yet, no one of the devices known at the state of the art allows to calculate a diagnostic parameter that considers explicitly also the frequency analysis of the associated raw ultrasound signal. It is to be specified that, also in the following, for raw ultrasonic signal it is intended a radiofrequency ultrasonic signal, as it is received by the probe, before the various filtrations and processing necessary for obtaining the ultrasound image.

### SUMMARY OF THE INVENTION

Aim of the present invention is to provide a device for carrying out an examination for breast cancer diagnosis, which overcomes the limits linked to the embodiments known at the state of the art, and in particular which allows to realize, during the same examination, a perfectly overlapping volumetric reconstruction both of the breast image and of the distribution of characteristic parameters of the analyzed tissues, such for example the acoustic attenuation coefficient.

According to another aim, the device object of the present invention comprises computing means on which computer programs are loaded to calculate a diagnostic parameter that considers explicitly both piece of information (information obtainable from the ultrasound imaging and information obtainable from the analysis of acoustic attenuation coefficients).

Yet, another aim of the present invention is to calculate a diagnostic parameter that considers explicitly also the parameters deriving from the frequency analysis of the ultrasound signals associated to specific portions of breast tissue. Description of the figures

### BRIEF DESCRIPTION OF THE DRAWINGS

FIGS. 1, 2 and 3 show three views of a preferred and not limiting embodiment of the device;

FIG. 4 shows a schematic view of a breast section along a generic plane of acquisition passing through the axis of symmetry of the device;

FIG. 5 shows a section schematic view of a portion of tissue along a plane orthogonal to the axis of symmetry of the device;

FIG. 6 shows a schematic view of a breast half section along a generic plane of acquisition passing through the axis of symmetry of the device, and FIG. 7 shows schematically: the shape of the signal received in transmission, in the time domain (a) and in the frequency domain (b), and the shape of the signal received in reflection, in the time domain (c) and in the frequency domain (d).

FIGS. 8 and 9 show examples of flowcharts of a preferred embodiment of the method which can be carried out by means of the device according to the invention;

FIG. 10 shows a schematic section view of a probe, highlighting the mechanic coupling means;

FIG. 11 shows the device installed under an examination table;

FIG. 12 shows a tridimensional schematization of the arrangement of various planes of acquisition.

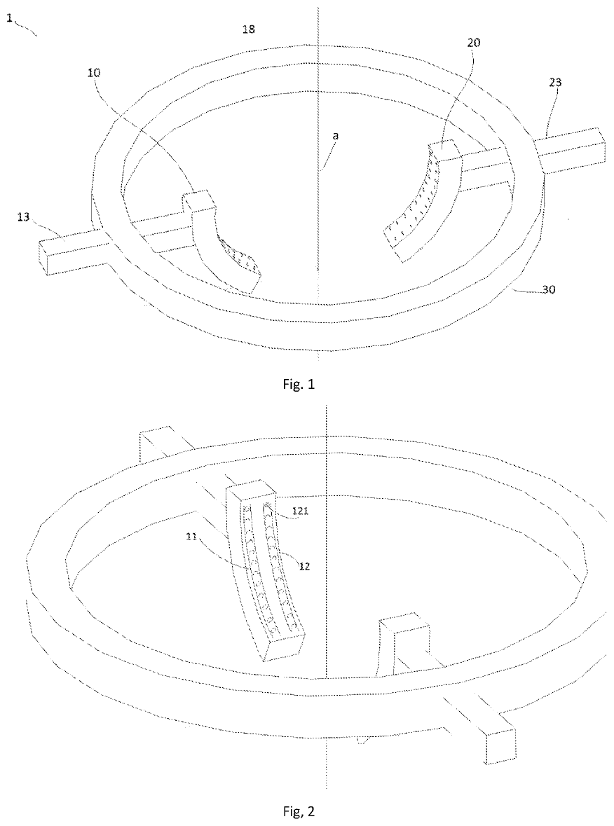

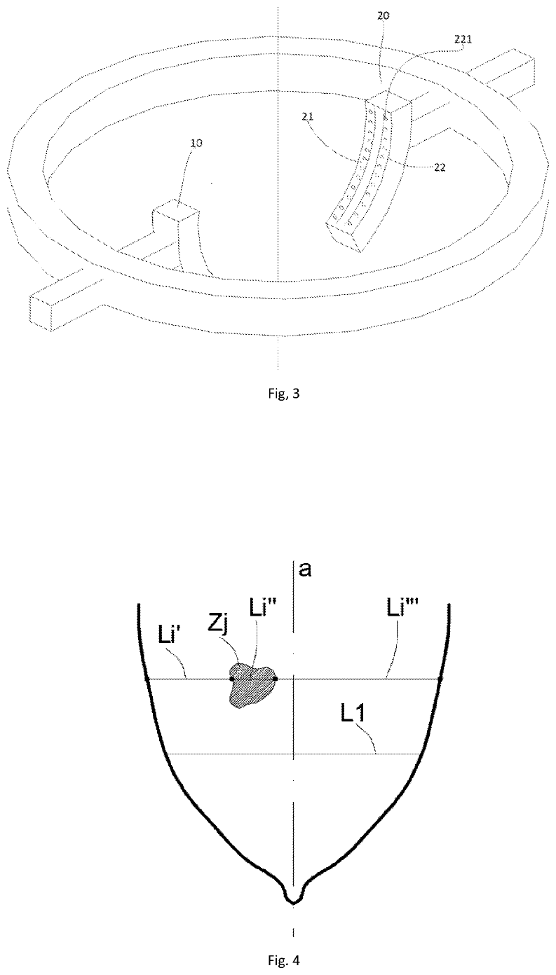

### DETAILED DESCRIPTION OF THE PREFERRED EMBODIMENTS

Before the following description, it is to be specified that: for tumor tissue it is intended the tissue relating to a suspect area for which the presence of a tumor has been verified by means of histologic examination or by means of the method according to the present invention;
for not tumoral tissue it is intended the tissue relating to a suspect area for which the presence of a tumor has been excluded by means of histologic examination or by means of the method according to the invention. In other words, the not tumoral tissue relates to an inhomogeneity detectable by ultrasound or by means of ultrasound analysis, resulted benign thereupon,
for healthy tissue it is intended the tissue with no inhomogeneity characteristics detectable by ultrasound or by means of ultrasound analysis, and not classified as suspect area.

It is also to be specified at first that the realization of the device is described with reference to the use of piezoelectric transducers, but other types of transducers can be also used, as for example CMUT capacitive transducers, without departing from the aims of the invention.

With reference to FIG. 1, the device (1) according to the invention comprises two ultrasound probes (10, 20), each one being associated to two arrays of piezoelectric transducers (11, 12, 21, 22). In a first embodiment, said arrays are linear arrays. In a second embodiment, said arrays are concave, so that the concavity allows a better breast shape adherence. It is to be specified that the concavity is associated to the outer shape of the probe head.

Each of the two ultrasound probes (10, 20) comprises a first array of piezoelectric transducers (11, 21) with a first nominal frequency or band center frequency (f1), and a second array of piezoelectric transducers (12, 22) with a second nominal frequency or band center frequency (f2).

Said first nominal frequency (f1) is chosen to carry out a B-Mode ultrasound imaging starting from the signals reflected by breast tissues and is preferably but not limitingly between 7 and 10 MHz. From the following description, it will be clear that each probe is used for high resolution ultrasound imaging acquisition of the breast portion comprised between the skin and the axis of symmetry thereof. For this reason, considering that the depth of the analyzed tissue is limited, high frequencies can be used without the attenuation associated thereto becomes a problem.

Said second nominal frequency (f2) is chosen to penetrate better the tissue, and so to allow acoustic attenuation measures working in transmission mode with the array of the other probe with the same nominal frequency. Said second nominal frequency (f2) is preferably between 1 and 3 MHz.

The two arrays (12, 22) with the lowest nominal frequency (f2) are provided with the same number of piezoelectric transducers, so that a respective transducer (221) of the array (22) of the second probe (20) corresponds to each transducer (121) of the array (12) of the first probe (10).

The realization of each of the two probes is such that all the piezoelectric transducers associated thereto are rigidly fastened to each other. In other words, the relative position of the two arrays of piezoelectric transducers is fixed. In particular, each probe comprises two arrays, linear or concave next to each other, of piezoelectric transducers.

Each probe comprises also coupling means (16, 26) to a control device (not shown in figure) configured to guide said probes and to detect and analyze the signal acquired by the same, according to what described in detail in the following.

In a first preferred embodiment, each probe (10, 20) comprises also a fixing rod (13, 23). The two fixing rods (13, 23) are associated to a circular support (30) in diametrically opposed positions. In other words, the two probes (10, 20) are positioned along the same direction, opposite to each other, with the arrays (11, 12, 21, 22) positioned in the same plane of the axis of symmetry (a) of the circular guide (30), and symmetrical to the same.

Each of the two probes (10, 20) is positioned along a radial direction of said circular guide (30), and it is associated in the same way so that the fixing rod (13, 23) can slide, thus allowing the probe (10, 20) to be positioned at different distances from the axis of symmetry (a) of the circular guide (30).

Preferably, the device comprises also thrusting means (not shown in FIG. 1) configured to thrust the two probes (10, 20) radially towards the center. Preferably, said thrusting means comprise at least a spring element, configured to thrust the respective probe (10, 20) towards the center.

The device comprises also movement means (40) configured to rotate said circular guide (30) around its own axis of symmetry (a).

The movement means (40) comprise one or more electric motors and respective control means. These are movement means known per se at the state of the art.

Regardless of the embodiment, the just described one being a preferred and not limiting variant thereof, the device (1) according to the invention comprises two ultrasound probes (10, 20), each comprising two arrays (11, 12, 21, 22) of piezoelectric transducers with two different nominal frequencies (f1, f2), said probes being arranged opposite to each other on the same straight line, and being configured so that they can rotate along a circular trajectory having for center the midpoint of the segment connecting them and so that they can slide to each other along the straight line connecting them.

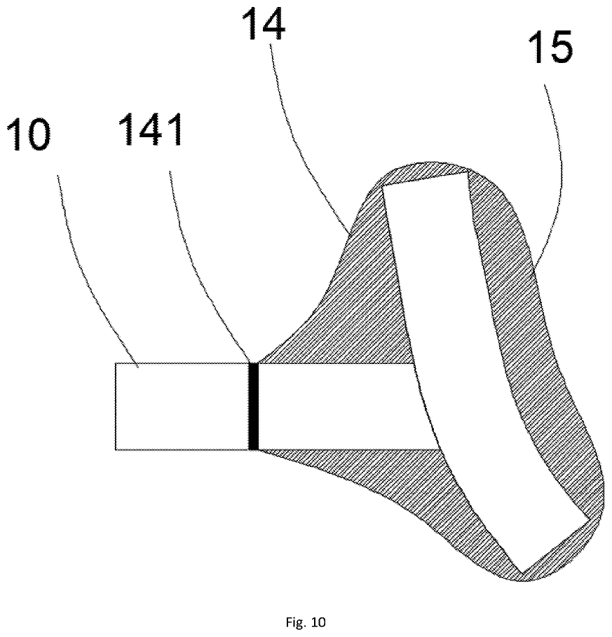

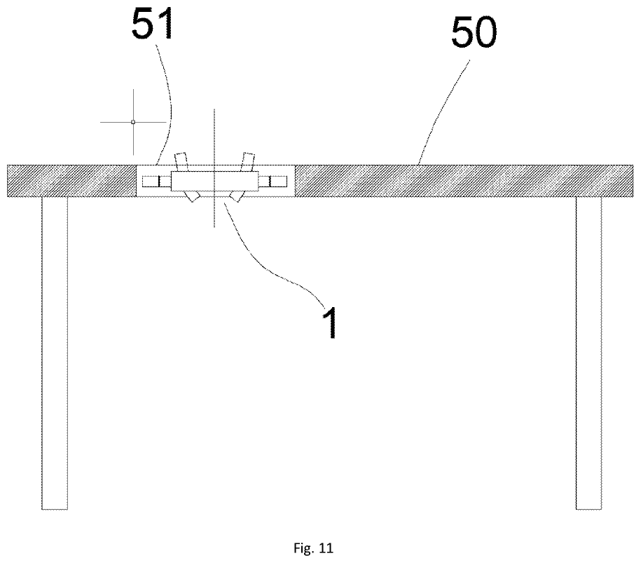

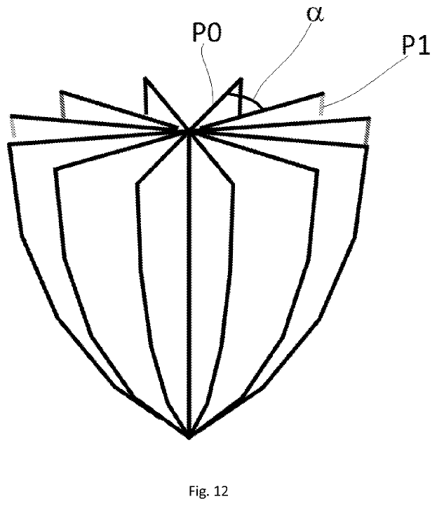

As it is shown in FIG. 10, moreover preferably each probe comprises also acoustic coupling means (14, 15) to the breast skin. In a first preferred embodiment, said acoustic coupling means comprise a flexible membrane (14), filled with a gel (15). The assembly of membrane and gel is configured to be pressed when the probe is thrusted towards the breast, thus adapting to the breast shape and thus guaranteeing the mechanic coupling with this latter for the ultrasound transmission. Preferably, to such aim the breast is covered with gel, liquid or any other material suitable for the ultrasound transmission.

In a second embodiment, the device is immersed in a vessel containing water or any other liquid suitable for functioning as acoustic coupling means, and open in the upper portion so that the breast can be introduced from above.

The device (1) is configured to be positioned under an examination table (50), at an opening (51) in which the breast of the woman, laying prone on the same table, can be introduced.

After describing the device, it is now possible to describe its functioning.

Once the patient is positioned prone on the table (51), the device (1) is positioned with its own axis of symmetry (a) at the axis of symmetry of the breast, and the two probes (10, 20) are thrusted towards the axis of symmetry, until they come in contact with the breast of the patient from diametrically opposed portions in a first angular position (P0).

According to another embodiment, the device can be fastened integrally to the lower portion of a table (51), at a hole obtained on the surface of the same in suitable position. Now, a first scanning can be carried out, according to what described in detail in the following, and so the circular guide (30) is rotated of an angle (a), up to bring the two ultrasound probes in position (P1), and the sequence of scanning and rotations is repeated up to complete a 180° rotation of the circular guide (30) and to come back with the two probes aligned in the first angular position (P0), but in inverted positions. In this way, the whole breast volume is scanned.

It is to be specified that the control device of the probes and acquisition of the ultrasound signals detected by them is configured: to guide individually each piezoelectric transducer of each array of each probe; to detect the signals acquired by each piezoelectric transducer of each probe;
to store the “raw” (also called “radiofrequency”) ultrasound signals acquired by each transducer, thus making them available for next processing; to process the radiofrequency signals for obtaining B-mode ultrasound images;
to associate the relative position of acquisition to each signal detected and to each image.

Clearly, for the just described procedure as well as for all the other ones described in the document, the device according to the invention comprises electronic computing means on which computer programs are loaded configured to carry out the relative movement procedures of the device, the data acquisition, data storing, data processing and diagnostic parameter calculation.

In each position (P0, P1, . . . ) the acquisition procedure is the following:
100) acquisition of a first B-mode ultrasound image relating to the portion of tissues comprised between the first probe (10) and the axis of symmetry, by means of the first array (11) of the first probe (10), by sending a plurality of ultrasound signals and detection of relative ultrasound signals reflected by the breast tissues;
110) acquisition of a second B-mode ultrasound image relating to the portion of tissues comprised between the second probe (10) and the axis of symmetry, by means of the first array (21) of the second probe (20), by sending a plurality of ultrasound signals and detection of the relative ultrasound signals reflected by the breast tissues; 120) storage of the “raw” (or “radiofrequency”) ultrasound signals, as detected by each probe (10, 20) and before any following processing, in particular before the processing for the relative B-mode image creation;
130) emission of an ultrasound pulse by means of the first piezoelectric transducer (121) of the second array (12) of the first probe (10), and detection of the relative ultrasound signal transmitted by means of the first piezoelectric transducer (221) of the second array (22) of the second probe (20) and the relative ultrasound signal reflected by means of the first piezoelectric transducer (121) of the second array (12) of the first probe (10);
140) repetition of step 130) for each couple of piezoelectric transducers of the second arrays (12, 22) of the first and second probe.

At the end of the acquisition procedure, for each angular position (P0, P1, . . . ), for the following processing are then available: two ultrasound images, acquired in the same plane, and relating to the portions of tissue (P0 1, P0 2) comprised between the axis of symmetry and each probe, obtained from the analysis of the reflected signals acquired by each of the two arrays having the first nominal frequency;
a plurality of couples of signals, made up of an emitted signal (which is known on the basis of the structural characteristics of the probe used and relative guide system, commonly known also as “beamformer”) and of a signal received in transmission mode, each couple of signals relative to a couple of transducers of the arrays of the first and second probe having the second nominal frequency;
a plurality of ultrasound signals reflected in the time domain acquired by the arrays of each probe having the second nominal frequency; a plurality of ultrasound signals in the time domain (110S), reflected by the breast tissues upon the pulses sent by the arrays having the first nominal frequency and associated to respective propagation segments (110).

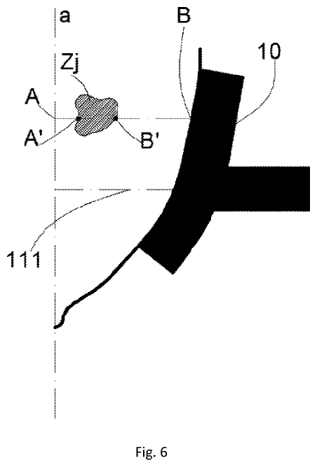

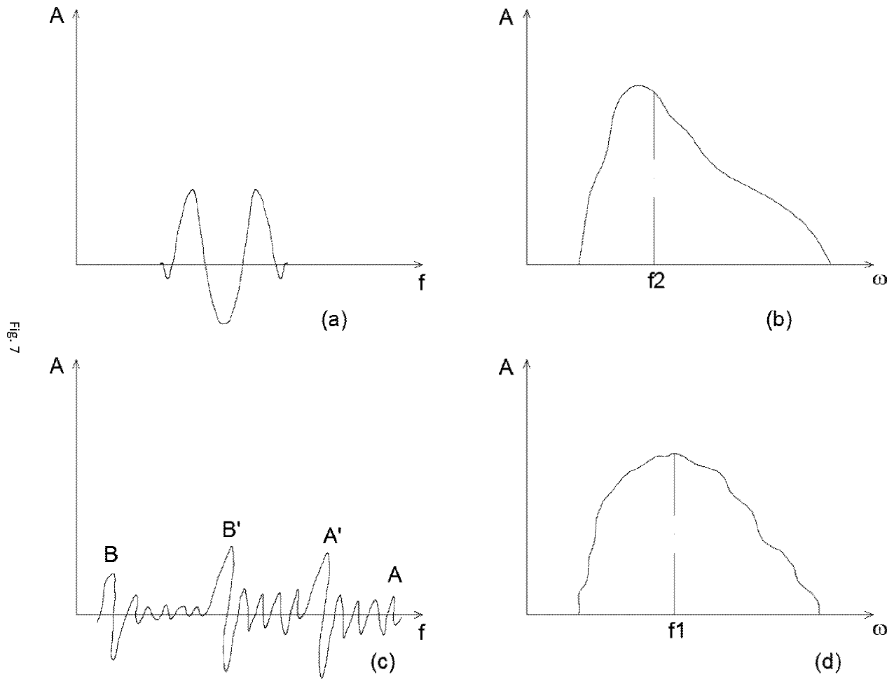

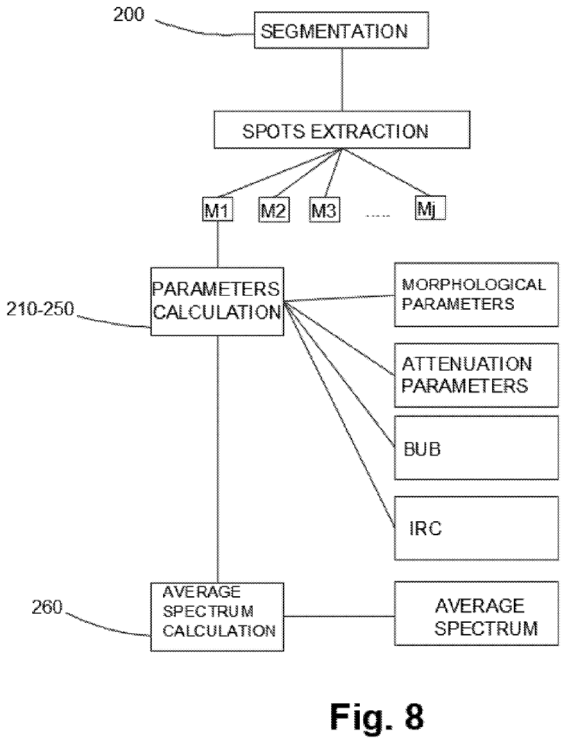

In FIG. 6, it is shown the path (110) of the ultrasound reflected signal detected by the first transducer of the first array (11) of the first ultrasound probe (10). For schematic purpose, FIG. 6 shows only the portion of tissue comprised between the axis of symmetry and the probe (10).

As it is always clear from the analysis of FIG. 6, which shows also the ultrasound reflected signal, to point A, positioned at greater distance from the probe, a signal corresponds that has an arrival time (tA) greater than the arrival time (tB) of the signal reflected from point B substantially arranged at the skin, in contact with the probe.

On the basis of this consideration, known per se, it is possible to associate the value of the corresponding reflected, raw (i.e. not processed) ultrasound signal to each point of the segment AB.

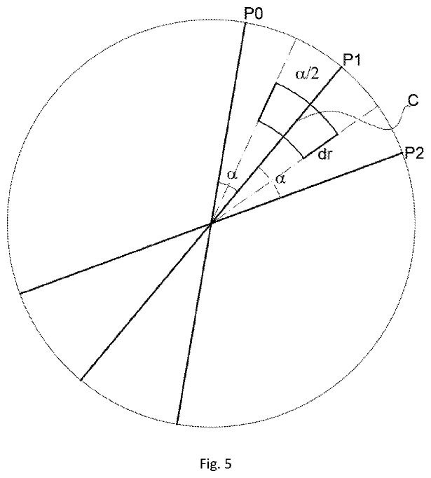

The assembly of the acquisitions carried out defines a volumetric grid of acquisition volumes, schematically shown in FIG. 5, where it is shown the plan projection of the ith (Vi) acquisition volume, associated to a generic point C.

So, the ith volume will have an angular amplitude (alfa) equal to the angle between two following positions of acquisition, a radial amplitude (dr) equal to the spatial resolution of the ultrasound imagining system in radial direction, i.e. in the sense of depth with respect to the ultrasound probe and a height (not shown in FIG. 5) corresponding to the “side spatial resolution” of the ultrasound image.

As it is known, the resolution of the ultrasound imagining system in the sense of depth corresponds theoretically to the half wave-length of the incident ultrasound pulse, which is inversely proportional to the frequency. It is also to be specified that the angular amplitude of the volume acquired depends on the focusing of the used ultrasound beam. Conveniently, the angle (a} between two following positions of acquisition (P0, P1) is chosen so that the whole volume is under acquisition and no areas are left uncovered.

So, to each ith volume, downwards of the acquisition a value (Si) relating to the intensity of the reflected ultrasound image,
a value (B±) of the grayscale, associated to the B-mode ultrasound image will be associated.

Moreover, the values of the acoustic attenuation coefficient calculated for each propagation line of the ultrasound signals emitted by said second array (12) of the first probe (10) and received by said second array (22) of the second probe (20); the acquisitions of the radiofrequency raw ultrasound signals, relating to all the propagation lines of the ultrasound signal of each array, for all the positions of acquisition are available.

With the previously enlisted data, the method of analysis of the same to calculate a diagnostic parameter representing the presence of a breast cancer or not comprises the steps of:
200) segmentation of the breast volume 3d model to individuate the presence of one or more “suspect areas”, characterized by clearer color (hyperechoic) or darker color (hypoechoic) with respect to the surrounding tissue, or anyway characterized by any other “inhomogeneity” form detectable by means of the grayscale analysis, which makes these areas “encirclable”.

In case of individuation of one or more suspect areas (Zj), calculation of a diagnostic parameter (Dj) referred to each area, indicating the probability that such area can be classified as breast cancer, said diagnostic parameter (Dj) depending on one or more of the following factors:
(i) morphological information deriving from the B-mode imaging of the points of the 3D model inside the ith area (Zj);
(ii) information relating to the average acoustic attenuation coefficient relative to at least a portion of propagation line of the ultrasound signal contained inside the jth area (Zj);
(iii) information relating to the frequency spectrum of the signal associated to the jth area (Zj) or anyway derived by the analysis thereof, possibly carried out by means of quantitative comparisons with “model spectra” acquired on patients with known diagnoses.

It is to be specified that, with reference to FIG. 4 (in which, for simplicity the propagation lines of the ultrasound signal are shown, but not the respective probes), the average acoustic attenuation coefficient relative to at least a portion of propagation line of the ultrasound signal contained in the jth area (Zj) can be calculated: by calculating the acoustic attenuation coefficient of the healthy tissue (Ats), i.e. relative to a propagation line of the ultrasound signal (of LI length) outside any suspect area individuated in the ultrasound image. Preferably, the acoustic attenuation coefficient of the healthy tissue (Ats) is calculated as the average of the acoustic attenuation coefficient calculated for a plurality of propagation lines of the ultrasound signal outside any suspect area detected during the ultrasound examination.
So, for each propagation line (with whole length Li) passing through the jth area (Zj), it is possible to calculate the average acoustic attenuation coefficient relative to the inner tissue of the suspect area (Aj), considering that the whole acoustic attenuation calculated for the ith line (Ai) is given by the sum of the acoustic attenuation along the healthy portions (of Li′ and Li″ length) and of the acoustic attenuation along the portion contained inside the suspect area (of Li′″ length), according to the following relation, in which the only unknown factor is the value of the average acoustic attenuation coefficient relative to the inner tissue of the suspect area (Aj), since the average acoustic attenuation coefficient along the whole ith propagation line (Ai) can be calculated considering the propagation line as it would be crossing an homogeneous tissue: Ats×(Li′+Li″)/(Li)+Aj×Li′″/Li=Ai

It is to be precised that the measurement of the lengths of the outer and inner segment of the suspect area (Li′, Li″, Li′″) is made possible only by the acquisition of an ultrasound image carried out in the same position of acquisition, and with a frequency greater than the ultrasound signal, in order to improve the resolution of the ultrasound image, and so the precision of the calculation of the lengths of the outer and inner segment of the suspect area (Li′, Li″, Li′″). In other words, this measurement is made possible only by the configuration of the arrays of piezoelectric transducers of the device according to the invention, which comprises two ultrasound probes opposite to each other, each one comprising two arrays with different nominal frequencies, the one optimized for the acquisition of a high resolution ultrasound image and the other one for the calculation of the acoustic attenuation coefficient, and generally for the execution of acoustic measurements in transmission at lower frequencies than the ones needed for obtaining good ultrasound images. In a preferred embodiment, for the calculation of the diagnostic parameter, the method can be carried out according to the following steps.
210) calculation of a plurality of morphological and intensity characteristics deriving from the ultrasound imaging of the jth area (Zj), among which for example:
   volume of the area;
   surface to volume ratio;
   maximum dimension;
   parameters of shape, such as eccentricity; —ratio between the average grey value of the jth area (Zj) and the average grey value of the surrounding region.

Preferably, said surrounding region is defined as the assembly of all the portions of breast tissue not belonging to any suspect area or as the assembly of all the portions of breast tissue not belonging to any suspect area and crossed by the same propagation lines of the considered jth area (Zj),
220) calculation of the average acoustic attenuation coefficient (Aj) relative to at least a portion of propagation line of the ultrasound signal contained inside the jth area (Zj);
230) calculation of the ratio between the average acoustic attenuation coefficient (A) of the jth area (Zj) and average acoustic attenuation coefficient of the outer region to the jth area (Ats),
235) calculation of the acoustic propagation speed of the ultrasound signal relating to at least a propagation line of the ultrasound signal passing through the jth area (Zj) and at least a propagation line of the ultrasound signal not passing through any suspect area.

Conveniently, the propagation speed, which gives an indication of the tissue density, can be calculated as a function of the receiving time of the ultrasound signal transmitted and of the relative distance between the probes (both measured automatically by the device according to the present invention). 240) calculation of the BUB (Broadband Ultrasound Backscatter) relative to the assembly of the 3d model points inside the jth area (Zj);

It is to be specified that the BUB is the average of the backscattering coefficient (which is a parameter depending on the frequency) in a determined frequency interval, preferably between 0.2 and 0.6 MHz or anyway in a frequency interval in which the spectrum of the reflected signal has a decreasing development. The backscattering coefficient can be calculated as the ratio between the intensity of the ultrasound signal reflected by the portion of tissue considered and a reference ultrasound signal. The signal reflected by the air-water interface positioned at the same distance from the probe with respect to the portion of tissue in the examination step can be considered as reference ultrasound signal. So, it is clear that the reference ultrasound signal is a parameter characterizing the probe once the parameters of the transmitted signal (frequency, power, etc.). and the reference distance are fixed.
250) calculation of the IRC (Integrated Reflection Coefficient) relative to the interface between the jth area (Zj) and the surrounding tissue.

IRC can be calculated as the average of the energy reflection coefficient in a determined frequency interval, preferably in the interval between 0.2 and 0.6 MHz, or anyway in a frequency interval in which the spectrum of the reflected signal has a decreasing development, and is obtained as logarithmic difference between the spectrum of the signal reflected by said interface in the analysis step (between the jth area and the surrounding tissue) and the signal reflected by the air-water reference interface (stored in the system in the construction step of the device and available for the calculation). Downwards of point 250), so it is defined a set of values of each parameter relative to each ozone (Zj).
260) calculation of at least a frequency spectrum of the radiofrequency raw ultrasound signal emitted by one of said first arrays (11, 21) and reflected by a segment of a propagation line of the ultrasound signal contained inside the jth area. 270) calculation of at least a quantitative parameter extracted by the spectrum of point 260), included in the list comprising the following parameters
   spectrum average value;
   area subtended by the spectrum in a determined frequency interval; spectrum width (intended as the difference between maximum and minimum frequency) at a predetermined intensity level, in particular at a level defined by an intensity value lower than the maximum value of said spectrum for a predetermined amount, in particular lower than 1 dB or 3 dB;
   the frequency value corresponding to the maximum value of said spectrum;
   the slope of a line interpolating a plurality of points of said spectrum in a predetermined frequency interval; the coefficients of a polynomial interpolating the points of said frequency spectrum in a frequency interval containing the maximum of said spectrum.

FIG. 6 shows the portion (110′) of the propagation line (110) of the ultrasound signal emitted by a piezoelectric crystal of the first array (11) of the first probe (10) comprised inside the jth area (Zj). FIG. 7 shows the relative ultrasound signal acquired in reflection in the time domain, and in particular the portion (A′-B′) associated to the portion of the propagation line contained inside the jth area (Zj). FIG. 7 shows also the spectrum (SI) obtained as frequency transform of the ultrasound signal reflected by the tissue comprised inside the jth area (Zj).

Preferably, the frequency spectrum associated to the jth area (Zj) is calculated as the average spectrum of a plurality of spectra associated to a plurality of propagation segments of the signal contained inside the jth area. Preferably, for the calculation of the average spectrum a selection step of the ultrasound signals is also carried out, to be used for the calculation of the average spectrum relative to the jth area;
calculation of the correlation coefficient of the average spectrum with all the spectra used for its calculation; exclusion of the spectra for which such correlation coefficient is lower than a predetermined threshold, for example 0.9;
calculation of a new average spectrum relative to the jth area; —repetition of the calculation procedure of the spectra correlation and exclusion, until all the spectra left have a correlation coefficient with the average spectrum greater than or equal to said predetermined threshold.
280) Calculation of a diagnostic parameter which is a function of the comparison of at least one of the parameters calculated in steps 210, 220, 230, 235, 240, 250, 260, 270, with the respective parameters relating to tumors detected in ultrasound examinations carried out on patients for whom the presence of a breast cancer has been subsequently confirmed by means of histologic examination; the respective parameters relating to suspect tumors detected in ultrasound examinations carried out on patients for whom the presence of a breast cancer has been subsequently excluded by means of a biopsy or other equivalent reliable technique;
   the respective parameters relative to portions of tissue not interested by cancers, detected in ultrasound examinations carried out on patients for whom the presence of a breast cancer has been subsequently excluded.

So, it is clear that the comparison can occur only after execution of examinations by means of the method according to the invention on a statistically significant number on patients, for whom the possible presence of a tumor has been subsequently confirmed by other tests, and that a data set is then available, acquired according to what just described and relative to a set of suspect areas (Zt), whose tumoral nature has been confirmed, to a set of suspect areas (Znt), whose tumoral nature has been excluded, and to a set of portions of tissues not interested by ultrasound visible suspect areas (Tnz).

Therefore, the comparison occurs downwards of the following procedure for reference values definition for the parameters.
300) Definition, for each parameter calculated at points 210 to 250, of an average tumoral value (VMt), and a relative confidence interval at 75% and 95%, calculated starting from the statistic distribution of the values of each parameter for all the suspect areas belonging to said set of suspect areas (Zt) whose tumoral nature has been confirmed;
310) definition, for each of the parameters calculated at points 210 to 250, of a not tumoral average value (VMnt), and a relative confidence interval at 75% and 95%, calculated starting from the statistical distribution of the values of each parameter for all the suspect areas belonging to said set of suspect areas, whose tumoral nature (Znt) has been excluded;
320) definition, for each parameter calculated at points 210 to 250, of a healthy average value (VMs), and a relative confidence interval at 75% and 95%, calculated starting from the statistic distribution of the values of each parameter for all the portions of tissues belonging to said set of portions of tissues not interested by ultrasound visible suspect areas (Tnz).

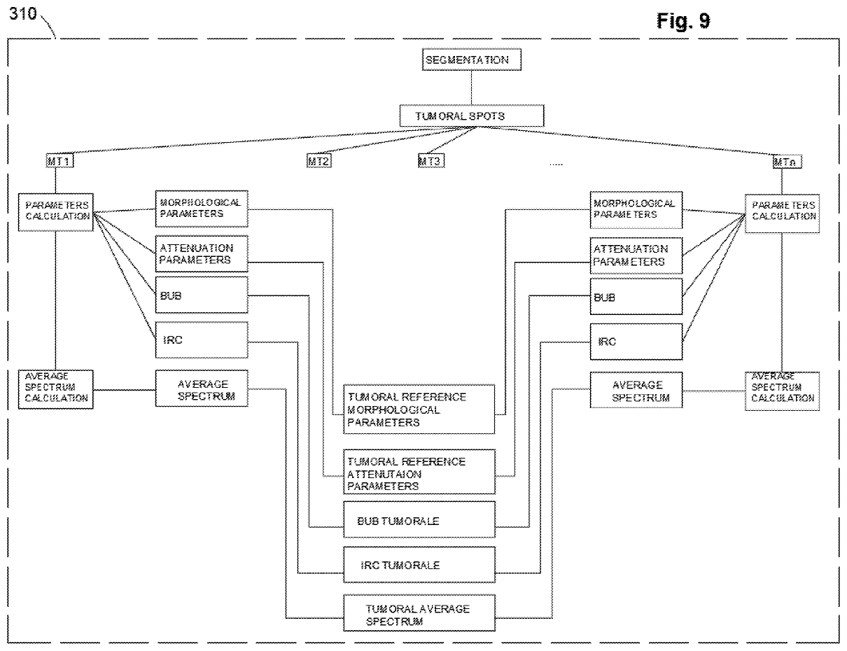

Downwards of point 320), three reference sets of values of each parameter are then defined: a first set relative to tumoral areas, a second set relative to suspect areas, which then result not tumoral, a third set relative to a tissue not belonging to suspect areas (healthy tissue). With reference to the calculation of the tumoral reference values, the step 310) is shown in the flowchart of FIG. 9, where it is observed as for all the tumoral spots (XT1, . . . ZTn) all the parameters (morphological, of acoustic attenuation, relative to BUB and IRC and relative to frequency spectra) are calculated, and for each parameter is then calculated the average of all the values calculated for the single tumoral areas to obtain a set of tumoral reference values comprising an average value for each parameter.

The procedure is the same for the not tumoral suspect areas and for the tissue interested by ultrasound not visible suspect areas.

The method can possibly provide the following selection step of significant parameters.
330) selection among the parameters of the reference sets of the ones for which the average tumoral value (VMt) is outside both the confidence interval at 75% of the relative healthy average value (VMs) and the confidence interval at 75% of the relative not tumoral average value (VMnt).

The definition procedure of the reference values comprises also the calculation of a series of reference frequency spectra.
340) selection, from the data acquired in ultrasound examinations carried out on patients for whom the breast cancer presence/absence diagnosis has been confirmed, of a plurality of segments of ultrasound propagation:
   contained inside tumoral areas; contained inside suspect areas, subsequently resulted not tumoral; —contained inside the outer tissue of the suspect areas;
350) calculation of the frequency transform of the radiofrequency raw ultrasound signal associated to each of said segments of ultrasound propagation;
360) calculation:
   of the tumoral reference spectrum obtained as the average spectrum relative to all the ultrasound signals relating to tumoral areas;
   of the not tumoral reference spectrum obtained as the average spectrum relative to all the ultrasound signals relating to not tumoral areas;
   of the reference spectrum relative to the outer tissues of the suspect areas obtained as the average spectrum relative to all the ultrasound signals relating to the outer tissue of the suspect areas.

Moreover, preferably the method comprises a selection step of the ultrasound signals to be used for the calculation of the reference spectra, defined as follows:
370) for each of the reference spectra calculated at point 360:
   calculation of the correlation coefficient of the reference spectrum with all the spectra used for its calculation; exclusion of the spectra whose correlation coefficient is lower than a predetermined threshold, for example 0.9;
   calculation of a new reference spectrum;
   repetition of the calculation procedure of the spectra correlation and exclusion, until all the spectra left have a correlation coefficient with the reference spectrum greater than or equal to said predetermined threshold.

Once the reference values of the parameters and the value of the reference frequency spectrum are known, a diagnostic parameter indicating the probability that the jth area (Zj) is tumoral or not is calculated.

In a first embodiment, said diagnostic parameter is a classification of the jth suspect area (Zj) as tumoral or not tumoral. In order to classify the jth area, the procedure is the following;
400) calculation of the correlation coefficient of the set of parameters relative to the jth area,
   with the reference set relative to tumoral areas,
   with the reference set relative to suspect areas, subsequently resulted not tumoral;
   with the reference set relative to tissue not belonging to suspect areas. 410) In case one of the three coefficients calculated at point 400 is greater than a first predetermined threshold (for example r>0.9) and the remaining ones are lower than a second predetermined threshold (for example r<0.7), classification of the jth area as tumoral if the correlation coefficient is greater than the one with the reference set relative to tumoral area, as not tumoral if it is one of the other two;
420) in case no one of the correlation coefficients is greater than said first predetermined threshold, or in case the two lower correlation coefficients are both not lower than said second predetermined threshold, the jth area is not classified.

In another embodiment, for the classification of the jth area, the procedure is the following:
500) calculation of the correlation coefficient of the average spectrum relative to the jth area with—the reference spectrum of a tumoral area; the reference spectrum of a suspect area, subsequently resulted not tumoral; the reference spectrum relative to the outer tissues of the suspect areas.
510) In case one of the three coefficients calculated at point 500 is greater than a first predetermined threshold (for example r>0.9) and the remaining ones are lower than a second predetermined threshold (for example r<0.7), classification of the jth area as tumoral if the correlation coefficient is greater than the one with the tumoral reference spectrum, as not tumoral if it is one of the other two;
520) in case no one of the correlation coefficients is greater than said first predetermined threshold, or in case the two lower correlation coefficients are both not lower than said second predetermined threshold, the jth area is not classified.

In another embodiment, for the classification of the jth area, the procedure is the following:
600) subdivision of the reference data sets available in a training data set and a validation data set,
610) training of a classification neural network, by using said training set, to classify a data set relative to a tumoral or not tumoral suspect area, 620) presentation of the data set relative to the jth area to the trained network,
630) classification of the jth area as tumoral or not tumoral as a function of the network output.

In a second embodiment, said diagnostic parameter is a numerical value of the probability that said jth suspect area (Zj) is tumoral or not tumoral. Said numerical value of the probability that the jth suspect area (Zj) is tumoral or not can be calculated by converting in percentage value said correlation coefficient of the average spectrum relative to the jth area with the reference spectrum of a tumoral area calculated at point 500).

1. An ultrasound device (1) for carrying out a breast diagnostic examination for breast cancer diagnosis comprising two ultrasound probes (10, 20), characterized in that each of said probes (10, 20) comprises a first array (11, 21) of piezoelectric or CMUT transducers with a first nominal frequency (f1) and a second array of piezoelectric or CMUT transducers (12, 22) with a second nominal frequency (f2), said probes (10, 20) being arranged opposite to each other, being configured to rotate along a circular trajectory having for center the midpoint of the segment connecting them and to slide to each other along said straight line connecting them, so that they come in contact with the breast of the patient from diametrically opposite portions, in that said arrays (11, 12, 21, 22) comprise each a plurality of piezoelectric or CMUT transducers, positioned so that acquisitions are carried out on a plane of acquisition orthoqonal to the plane of said circular trajectory, and in that 38 said device is configured to carry out, by moving said probes (10, 20) along said circular trajectory, a plurality of scans relative to a plurality of planes of acquisition (P0, P1 . . . ) passing through said midpoint, orthogonal to said circular trajectory and rotated to each other of an angle (a).

2. The ultrasound device (1) for carrying out a breast diagnostic examination for breast cancer diagnosis, according to claim 1, wherein said device comprises also a circular support (30), and in that each probe (10, 20) comprises also a fixing rod (13, 23) associated to said circular support (30), so that the two probes are positioned along the same direction, opposite to each other, with the arrays (11, 12, 21, 22) positioned in the same plane of the axis of symmetry (a) of said circular guide (30).

3. The ultrasound device (1) for carrying out a breast diagnostic examination for breast cancer diagnosis, according to claim 1, wherein the shape of the head of each of said probes (10, 20) is concave, so that a better breast shape adherence is allowed.

4. The ultrasound device (1) for carrying out a breast diagnostic examination for breast cancer diagnosis, according to claim 1, wherein said first nominal frequency (f1) is chosen to carry out a B-Mode ultrasound imaging starting from the signals reflected by the breast tissues, and said second nominal frequency (f2) is chosen to carry out acoustic attenuation measures, working in transmission mode with the array of the other probe having the same nominal frequency.

5. The ultrasound device (1) for carrying out a breast diagnostic examination for breast cancer diagnosis, according to claim 1, wherein said first nominal frequency (fl) is between 7 and 10 MHz and said second nominal frequency (f2) is between 1 and 3 MHz.

6. The ultrasound device (1) for carrying out a breast diagnostic examination for breast cancer diagnosis, according to claim 1, wherein each probe comprises also acoustic coupling means (14, 15) with the breast skin, comprising a flexible membrane (14), filled with a gel (15), the assembly of membrane (14) and gel (15) being configured to be pressed when the probe is thrusted towards the breast, thus adapting to the breast shape and thus guaranteeing mechanic coupling for ultrasound transmission.

7. The ultrasound device (1) for carrying out a breast diagnostic examination for breast cancer diagnosis, according to claim 1, further comprising electronic computing means on which computer programs are loaded configured to carry out, for each position of acquisition, the following procedure of acquisition:
100) acquisition of a first B-mode ultrasound image relating to the portion of breast tissues comprised between said first probe (10) and said axis of symmetry, by means of said first array (11) of said first probe (10);
110) acquisition of a second B-mode ultrasound image relating to the portion of tissues comprised between said second probe (10) and said axis of symmetry, by means of said first array (21) of said second probe (20); 120) storage of “raw” reflected ultrasound signals, as detected by each probe (10, 20) before any further processing, in particular before the processing for the corresponding B-mode image creation,
130) emission of an ultrasound pulse by means of the first piezoelectric or CMUT transducer (121) of said second array (12) of said first probe (10), and detection:
of the relative ultrasound signal transmitted by means of the first piezoelectric or CMUT transducer (221) of said second array (22) of said second probe (20) and
of the relative ultrasound signal reflected by means of said first piezoelectric or CMUT transducer (121) of said second array (12) of said first probe (10),
140) repetition of step 130) for each couple of piezoelectric or CMUT transducers of said second arrays (12, 22) of said first (10) and second probe.

8. The ultrasound device (1) for carrying out a breast diagnostic examination for breast cancer diagnosis, according to claim 7, is configured to carry out a plurality of acquisitions 42 in a series of acquisition planes (P0, P1), rotated to each other of a predetermined angle (a), so that to each point of each plane of acquisition is associated: a value (Si) relating to the intensity of the reflected ultrasound signal,
a value (Bi) of the grayscale, associated to the B-mode ultrasound image, the group of said values (Bi) thus constituting a 3d ultrasound model of the breast volume, and in that for each position of acquisition are stored: raw radiofrequency reflected ultrasound signals relating to all the propagation lines of the ultrasound signal of each first (11, 21) and second array (12, 22), raw radiofrequency transmitted ultrasound signals, relating to all the propagation lines of the ultrasound signal of said second arrays (12, 22).

9. The ultrasound device (1) for carrying out a breast diagnostic examination for breast cancer diagnosis, according to claim 8, wherein said computer programs are configured to carry out a method for calculating a diagnostic parameter 43 representing the presence or not of a breast cancer comprising the steps of:
200) segmentation of said 3d ultrasound model of the breast volume to individuate the presence of one or more suspect areas (Zj), characterized by brighter color (hyperechoic) or darker color (hypoechoic) with respect to the surrounding tissue, or anyway characterized by any other inhomogeneity detectable by means of grayscale analysis,
201) calculation of a diagnostic parameter (Dj) referred to each suspect area (Zj), indicating the probability that such area can be classified as breast cancer, said diagnostic parameter (Dj) depending on one or more of the following factors:
(i) morphological information deriving from the B-mode imaging of the points of said 3D model inside the ith area (Zj);
(ii) information relating to the average acoustic attenuation coefficient relative to at least a portion of propagation line of the ultrasound signal contained inside the jth area (Zj 44);
(iii) information relating to the frequency spectrum of the signal associated to the jth area (Zj).

10. The ultrasound device (1) for carrying out a breast diagnostic examination for breast cancer diagnosis, according to claim 9, wherein said diagnostic parameter is calculated by means of the method according to the following steps of:
210) calculation of a plurality of morphological characteristics deriving from the ultrasound imaging of the jth area (Zj), chosen from the list below:
volume of the area;
surface to volume ratio;
maximum dimension;
eccentricity;
ratio between the average grey value of the jth area (Zj) and the average grey value of the surrounding region;
220) calculation of the average acoustic attenuation coefficient (Aj) relative to at least a portion of propagation line of the ultrasound signal contained inside the jth area (Zj); 45
230) calculation of the ratio between said average acoustic attenuation coefficient and the average acoustic attenuation coefficient of the outer region to the jth area (Ats);
235) calculation of the acoustic propagation speed of the ultrasound signal relating to at least one propagation line of the ultrasound signal passing through the jth area (Zj) and to at least one propagation line of the ultrasound signal not passing through any suspect area;
260) calculation of at least one frequency spectrum of the radiofrequency raw ultrasound signal emitted by one of said first arrays (11, 21) and reflected by a tissue portion corresponding to a segment of a propagation line of the ultrasound signal contained inside the jth area;
270) calculation of at least one quantitative parameter extracted by the spectrum of point 260), chosen from the list comprising the following parameters:
spectrum average value;
area subtended by the spectrum in a determined frequency interval and/or determined intensity interval; spectrum width in a predetermined frequency interval;
frequency value corresponding to the maximum value of said spectrum;
slope of a line interpolating a plurality of points of said spectrum in a predetermined frequency interval;
coefficients of a polynomial interpolating the points of said spectrum in a frequency interval containing the maximum of said spectrum;
280) calculation of a diagnostic parameter function of the comparison of at least one of the parameters calculated in steps 210 to 270 with respective parameters obtained by means of ultrasound examinations carried out by means of the device according to any one of the preceding claims and referred to:
suspect areas for which the presence of the pathology has been subsequently confirmed by means of histologic examination; suspect areas for which the presence of a breast cancer has been subsequently excluded by means of a biopsy or other equivalent reliable technique;
portions of tis sue not interested by suspect areas.

11. The ultrasound device (1) for carrying out a breast diagnostic examination for breast cancer diagnosis, according to claim 10, wherein at point 260) a frequency spectrum is calculated associated to the jth area (Zj) as the average of a plurality of spectra relative to respective propagation segments of the signal contained inside said suspect area (Zj), and in that for the calculation of the average spectrum the following spectra selection procedure is carried out, to be used for the calculation of the average spectrum relative to the jth area:
calculation of the correlation coefficient of the average spectrum with all the spectra used for its calculation; exclusion of the spectra for which such correlation coefficient is lower than a predetermined threshold;
calculation of a new average spectrum relative to the jth area;
repetition of the calculation procedure of the spectra correlation and exclusion, until all the spectra left have a correlation coefficient with the average spectrum greater than or equal to said predetermined threshold.

12. The ultrasound device (1) for carrying out a breast diagnostic examination for breast cancer diagnosis, according to claim 11, wherein said comparison of point 280) occurs downwards of the following definition procedure of tumoral, not tumoral and healthy reference value sets, detected in ultrasound examinations carried out by means of the device according to any one of the preceding claims on patients for whom the presence of the tumoral pathology has been subsequently confirmed or excluded by means of histologic examination: 300) definition, for each parameter calculated at points 210 to 250, of an average tumoral value (VMt), and of a relative confidence interval at 75% and 95%, calculated starting from the statistic distribution of the values of each parameter for all the suspect areas whose tumoral nature has been confirmed;
310) definition, for each of the parameters calculated at points 210 to 250, of a not tumoral average value (VMnt), and of a relative confidence interval at 75% and 95%, calculated starting from the statistical distribution of the values of each parameter for all the suspect 49 areas whose tumoral nature (Znt) has been excluded; 0) definition, for each parameter calculated at points 210 to 250, of a healthy average value (VMs), and of a relative confidence interval at 75% and 95%, calculated starting from the statistic distribution of the values of each parameter for all the portions of tissues belonging to said set of portions of healthy tissue not interested by ultrasound-visible suspect areas (Tnz); 0) selection of a plurality of segments of propagation of the ultrasound signal:
contained inside tumoral areas;
contained inside suspect areas, subsequently resulted not tumoral; contained inside tissue located outside of suspect areas; 0) calculation of the frequency transform of the radiofrequency raw ultrasound signal associated to each of said segments of propagation of the ultrasound signal; 0) calculation:
of the tumoral reference spectrum obtained as the average spectrum relative to all the ultrasound signals relating to tumoral areas; 50 of the not tumoral reference spectrum obtained as the average spectrum relative to all the ultrasound signals relating to suspect areas, subsequently resulted not tumoral; of the reference spectrum relative to the outer tissues of the suspect areas obtained as the average spectrum relative to all the ultrasound signals relating to the outer tissue of the suspect areas.

13. The ultrasound device (1) for carrying out a breast diagnostic examination for breast cancer diagnosis, according to claim 10, wherein said definition procedure of tumoral reference value sets comprises the step of:
330) selection of the parameters for which the average tumoral value (VMt) is outside both the confidence interval at 75% of the relative healthy average value (VMs), and the confidence interval at 75% of the relative not tumoral average value (VMnt).

14. The ultrasound device (1) for carrying out a breast diagnostic examination for breast cancer diagnosis, according to claim 10, wherein said diagnostic parameter is a classification of said jth suspect area (Zj) as “tumoral” or “not tumoral”, obtained by:
400) calculation of the correlation coefficient of the set of parameters relative to the jth area with the reference set relative to tumoral areas, with the reference set relative to suspect areas, resulted not tumoral in subsequent analyses; —with the reference set relative to tissue not belonging to suspect areas,
410) in case one of the three coefficients calculated at point 400) is greater than a first predetermined threshold and the remaining ones are lower than a second predetermined threshold, classification of the jth area as tumoral if the correlation coefficient is greater than the one with the reference set relative to tumoral areas, as not tumoral if it is one of the other two;
420) in case no one of the correlation coefficients is greater than said first predetermined threshold, or in case the two lower correlation coefficients are both not lower than said second predetermined threshold, the jth area is not 52 classified and further diagnostic examinations are recommended.

15. The ultrasound device (1) for carrying out a breast diagnostic examination for breast cancer diagnosis, according to claim 10, wherein said diagnostic parameter is a classification of the jth suspect area (Zj) as tumoral or not tumoral, obtained by:
500) calculation of the correlation coefficient of the average spectrum relative to the jth area with
the tumoral reference spectrum; the reference spectrum of a suspect area, subsequently resulted not tumoral;
the reference spectrum relative to the outer tissues of the suspect areas calculated at point 360);
510) in case one of the three coefficients calculated at point 500 is greater than a first predetermined threshold and the remaining ones are lower than a second predetermined threshold, classification of the jth area as tumoral if the correlation coefficient is greater than the one with the tumoral reference spectrum, as not tumoral if it is one of the other two;
520) in case no one of the correlation coefficients is greater than said first predetermined threshold, or in case the two lower correlation coefficients are both not lower than said second predetermined threshold, the jth area is not classified.

16. The ultrasound device (1) for carrying out a breast diagnostic examination for breast cancer diagnosis, according to claim 10, wherein said diagnostic parameter is a classification of the jth suspect area as tumoral or not tumoral, obtained by:
600) subdivision of the reference data sets available in a training data set and a validation data set,
610) training of a classification neural network, by using said training set, to classify a data set relative to a tumoral or not tumoral suspect area,
620) presentation of the data set relative to the jth area to the trained network,
630) classification of the jth area as tumoral or not tumoral as a function of the network output.

17. The ultrasound device (1) for carrying out a breast diagnostic examination for breast cancer diagnosis, according to claim 15, wherein said diagnostic parameter is a numerical value of the probability that said jth suspect area (Zj) is tumoral or not tumoral, calculated by converting in percentage value said correlation coefficient of the average spectrum relative to the jth area with the reference spectrum of a tumoral area calculated at point 500).
